(19) United States
(12) Patent Application Publication
(10) Pub. No.: US 2013/0165763 A1
(43) Pub. Date: Jun. 27, 2013

(54) AIR-PUFF TYPE INTRAOCULAR PRESSURE MEASURING DEVICE

(71) Applicants: Wen Wei HUANG, Taoyuan County (TW); Chung Ping CHUANG, Taoyuan County (TW)

(72) Inventors: Wen Wei HUANG, Taoyuan County (TW); Chung Ping CHUANG, Taoyuan County (TW)

(73) Assignee: CRYSTALVUE MEDICAL CORPORATION, Taoyuan County (TW)

(21) Appl. No.: 13/585,583

(22) Filed: Aug. 14, 2012

(30) Foreign Application Priority Data
Dec. 23, 2011 (TW) 100148209

(51) Int. Cl. A61B 3/16; A61B 6/02; A61B 6/00

(52) U.S. Cl. 600/401

(57) ABSTRACT

An air-puff type intraocular pressure measuring device includes an optical measuring unit and a puffing unit. The optical measuring unit includes an imaging optical path having a perforated lens and an image sensor capable of receiving an eyeball image via the perforated lens for eyeball alignment; a measuring optical path having a measuring element for transmitting a measuring signal and receiving a reflected signal via the perforated lens to derive an intraocular pressure value; and a beam splitter for the image sensor and the measuring element to respectively form a first and a second path having different axial directions. The puffing unit is connected to the optical measuring unit for puffing air through the perforated lens against an eyeball. The puffing unit has a puffing path located coaxially on the second path of the measuring optical path, so that measuring errors caused by parts-related tolerances are effectively reduced.

### FIELD OF THE INVENTION

The present invention relates to a measuring device for sending a puff of air of predetermined pressure against an examinee's eyeball, and more particularly to an intraocular pressure measuring device that includes a puffing path located coaxially on a measuring optical path.

### BACKGROUND OF THE INVENTION

There are various types of intraocular pressure measuring devices, and the most popular ones include applanation tonometers, ton opens and pneumatonometers. Among others, the applanation tonometers are the most reliable means for measuring the intraocular pressure. However, a topical anesthetic must be introduced onto the examinee's cornea before the applanation tonometer contacts with the cornea for measuring the intraocular pressure.

The tonopens are similar to the applanation tonometers in design principle because they make contact with the examinee's cornea. While the tonopens are conveniently portable for quick screening test, they have relatively higher failure rate and error rate.

When using a pneumatonometer, an amount of air of predetermined pressure is instantaneously puffed to an examinee's cornea to flatten a predetermined area of the latter, and then an electronic device is used to detect the change of the cornea by a reflected light wave for calculating a value of the examinee's intraocular pressure. An advantage of the pneumatonometer is that it does not contact with the examinee's cornea. However, measuring error occurs when the intraocular pressure is higher above 30 to 40 millimeters of mercury (mmHg). Thus, the pneumatonometer is mainly used in screening tests.

Please refer to FIG. 1. A conventional pneumatonometer includes a slit plate 11 located in front of an examinee's eyeball 10, a first lens 12 and a second lens 13 sequentially located behind the slit plate 11, and an image sensor 14 located behind the second lens 13, so that an imaging optical path 15 is formed. A puffing unit (not shown) is mounted between the first les 12 and the slit plats 11. A slit on the slit plate 11 functions as a nozzle, so that a puffing path 16 is formed thereat and air from the puffing unit is directly puffed against the examinee's eyeball 10 via the slit of the slit plate 11.

The conventional pneumatonometer has a measuring optical path 17, which includes an infrared light source 18 for projecting onto the eyeball 10 in a direction different from that of the puffing unit and a photoelectric cell 19 for receiving a signal reflected from the eyeball 10 to calculate a value of the examinee's intraocular pressure.

In the conventional pneumatonometer, since the measuring optical path and the puffing path are provided on two different paths, parts-related tolerances and errors in assembling parts tend to cause differences in measuring results. Therefore, it is desirable to improve the measuring optical path and the imaging optical path of the conventional pneumatonometer, so as to more accurately calculate the intraocular pressure value.

### SUMMARY OF THE INVENTION

A primary object of the present invention is to provide an air-puff type intraocular pressure measuring device that includes a puffing path located coaxially on a measuring optical path to effectively reduce measuring errors caused by parts-related tolerances.

Another object of the present invention is to provide an air-puff type intraocular pressure measuring device that uses optical coherence tomography (OCT) technique in measuring the position at where the examinee's cornea is flattened and the time needed to flatten the cornea, so as to effectively shorten the time needed for intraocular pressure measurement.

To achieve the above and other objects, the air-puff type intraocular pressure measuring device according to the present invention mainly includes an optical measuring unit and a puffing unit. The optical measuring unit includes an imaging optical path having a perforated lens and an image sensor, the perforated lens having a perforation and the image sensor being capable of receiving an examinee's eyeball image via the perforated lens for eyeball alignment; a measuring optical path having a measuring element for measuring intraocular pressure, the measuring element transmitting a measuring signal toward the perforation of the perforated lens and receiving a reflected signal via the perforation of the perforated lens to derive a current intraocular pressure value; and a beam splitter located on the imaging optical path and the measuring optical path for the image sensor of the imaging optical path and the measuring element of the measuring optical path to respectively form a first and a second path having different axial directions.

The puffing unit is connected to the optical measuring unit for supplying air thereto. An amount of the supplied air is puffed through the perforation of the perforated lens against the examinee's eyeball. The puffing unit has a puffing path located coaxially on the second path of the measuring optical path, but non-coaxially on the first path of the imaging optical path.

According to the present invention, at least one relay lens can be further provided in each of the imaging optical path and the measuring optical path. In an operable embodiment, a first relay lens is provided between the perforated lens and the beam splitter, a second relay lens is provided between the beam splitter and the image sensor, and a third relay lens is provided between the beam splitter and the measuring element.

In a preferred embodiment of the present invention, the measuring element can be an optical coherence tomography (OCT) device or a charge coupled device (CCD) sensor; and the image sensor can be a complementary metal-oxide-semiconductor (CMOS) image sensor.

The present invention is characterized in that the measuring optical path and the puff unit are coaxially located on the same path to effectively reduce measuring errors caused by parts-related tolerances; and that, by using of the OCT technique in measuring the position at where the cornea is flattened and the time needed to flatten the cornea, it is able to shorten the time needed for intraocular pressure measurement.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a conceptual view of an imaging optical path for a conventional intraocular pressure measuring device; and

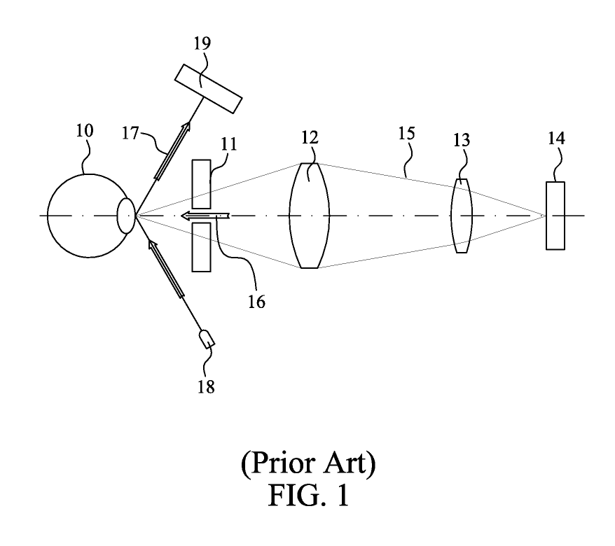

FIG. 2 is a conceptual view of an imaging optical path for an air-puff type intraocular pressure measuring device according to a preferred embodiment of the present invention.

### DETAILED DESCRIPTION OF THE PREFERRED EMBODIMENTS

The present invention will now be described with a preferred embodiment thereof and with reference to the accompanying drawings.

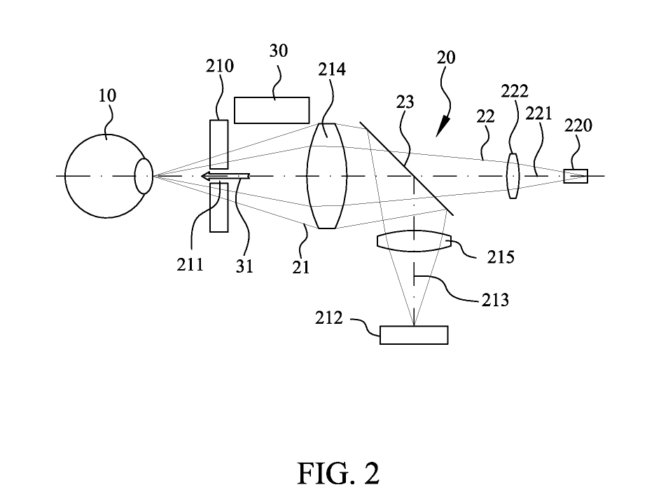

Please refer to FIG. 2. An air-puff type intraocular pressure measuring device according to a preferred embodiment of the present invention mainly includes an optical measuring unit 20 for measuring intraocular pressure of an eyeball 10, and a puffing unit 30 for puffing air against the eyeball 10.

In the preferred embodiment, the optical measuring unit 20 mainly includes an imaging optical path 21, a measuring optical path 22, and a beam splitter 23. The imaging optical path 21 is provided at an end adjacent to the eyeball 10 with a perforated lens 210 having a perforation 211, and at another end with an image sensor 212. The image sensor 212 receives the image of an examinee's eyeball 10 via the perforation 211 on the perforated lens 210, in order to align the intraocular measuring device with the examinee's eyeball 10.

The measuring optical path 22 includes an intraocular pressure measuring element 220, which transmits a measuring signal toward the perforation 211 of the perforated lens 210 and receives a signal reflected from the examinee's eyeball 10 and passing through the perforation 211 of the perforated lens 210, and computes the reflected signal to derive a current intraocular pressure value.

The beam splitter 23 is located on the imaging optical path 21 and the measuring optical path 22, so that the image sensor 212 of the imaging optical path 21 and the measuring element 220 of the measuring optical path 22 respectively form a first path 213 and a second path 221, which have different axial directions.

The puffing unit 30 is connected to the optical measuring unit 20 for supplying air to the latter, and the perforation 211 of the perforated lens 210 serves as a puffing path 31, via which an amount of the supplied air is puffed out against the examinee's eyeball 10.

The intraocular pressure measuring device according to the present invention is characterized in that the puffing path 31 of the puffing unit 30 is located coaxially on the second path 221 of the measuring optical path 22, but non-coaxially on the first path 213 of the imaging optical path 21. With the puffing path 31 located coaxially on the measuring optical path 22, it is able to effectively reduce measuring errors caused by parts-related tolerances.

According to the present invention, the imaging optical path 21 and the measuring optical path 22 can respectively include additional relay lenses. As can be seen in FIG. 2, in the illustrated preferred embodiment, a first relay lens 214 is provided between the perforated lens 210 and the beam splitter 23, a second relay lens 215 between the beam splitter 23 and the image sensor 212, and a third relay lens 222 between the beam splitter 23 and the measuring element 220; and the puffing unit 30 is connected to between the perforated lens 210 and the first relay lens 214.

However, it is understood the above arrangements are only illustrative and not intended to limit the type and the quantity of the lenses and mirrors in the imaging optical path 21 and the measuring optical path 22. That is, other different types and quantities of lenses and mirrors can be added to the imaging optical path 21 and the measuring optical path 22 according to actual functional requirements.

In a preferred embodiment, the measuring element 220 can be an optical coherence tomography (OCT) device or a charge coupled device (CCD) sensor; and the image sensor 212 can be a complementary metal-oxide-semiconductor (CMOS) image sensor.

In the present invention, with the image sensor 212 of the imaging optical path 21, a relative position between the examinee's eyeball 10 and the intraocular pressure measuring device can be detected and corrected. And then, with the puffing path 31 located coaxially on the measuring element 220, measuring errors caused by parts-related tolerances can be further minimized. Finally, the measuring element 220 directly computes the change volume of the flattened area of the examinee's cornea to derive the current intraocular pressure value of the examinee's eyeball 10.

According to the design of the present invention, since the measuring optical path and the puff unit are coaxially located on the same path, measuring errors caused by parts-related tolerances can be effectively reduced. Meanwhile, by using the OCT technique in measuring the flattened position on the cornea and the time needed to flatten the cornea, the time needed for intraocular pressure measurement is shortened.

The present invention has been described with a preferred embodiment thereof and it is understood that many changes and modifications in the described embodiment can be carried out without departing from the scope and the spirit of the invention that is intended to be limited only by the appended claims.

What is claimed is:

1. An air-puff type intraocular pressure measuring device, comprising:
an optical measuring unit including:
an imaging optical path having a perforated lens and an image sensor; the perforated lens having a perforation, and the image sensor receiving an image of an examinee's eyeball via the perforation of the perforated lens for aligning the examinee's eyeball with the intraocular pressure measuring device;
a measuring optical path including a measuring element for measuring the examinee's intraocular pressure; the measuring element transmitting a measuring signal toward the perforation of the perforated lens and receiving a signal reflected from the examinee's eyeball via the perforation of the perforated lens, and computing the reflected signal to derive a value of the examinee's current intraocular pressure; and
a beam splitter located on the imaging optical path and the measuring optical path for the image sensor of the imaging optical path and the measuring element of the measuring optical path to respectively form a first path and a second path, which have different axial directions; and
a puffing unit being connected to the optical measuring unit for supplying air to the latter; and the perforation of the perforated lens serving as a puffing path, via which an amount of the supplied air is puffed out against the examinee's eyeball;
wherein the puffing path is located coaxially on the second path but non-coaxially on the first path.

2. The air-puff type intraocular pressure measuring device as claimed in claim 1, further comprising at least one relay lens in each of the imaging optical path and the measuring optical path.

3. The air-puff type intraocular pressure measuring device as claimed in claim 2, wherein a first relay lens is provided between the perforated lens and the beam splitter, a second relay lens is provided between the beam splitter and the image sensor, and a third relay lens is provided between the beam splitter and the measuring element.

4. The air-puff type intraocular pressure measuring device as claimed in claim 3, wherein the puffing unit is provided between the perforated lens and the first relay lens.

5. The air-puff type intraocular pressure measuring device as claimed in claim 1, wherein the measuring element is an optical coherence tomography (OCT) device.

6. The air-puff type intraocular pressure measuring device as claimed in claim 1, wherein the measuring element is a charge coupled device (CCD) sensor.

7. The air-puff type intraocular pressure measuring device as claimed in claim 1, wherein the image sensor is a complementary metal-oxide-semiconductor (CMOS) image sensor.
